SEQUENCE LISTING (241 sequences)

SEQ ID NO: 1 - DNA, 22 bp, Artificial Sequence
  Description of Artificial Sequence Synthetic primer
aagttggaagtagggattgagt

SEQ ID NO: 2 - DNA, 23 bp, Artificial Sequence
  Description of Artificial Sequence Synthetic primer
caaaataaaataccaccctaaca

SEQ ID NO: 3 - DNA, 20 bp, Artificial Sequence
  Description of Artificial Sequence Synthetic primer
gtcggaagtagggattgagc

SEQ ID NO: 4 - DNA, 22 bp, Artificial Sequence
  Description of Artificial Sequence Synthetic primer
gaaataaaataccgccctaacg

SEQ ID NO: 5 - DNA, 21 bp, Artificial Sequence
  Description of Artificial Sequence Synthetic primer
tttttaggttttagggttggt

SEQ ID NO: 6 - DNA, 22 bp, Artificial Sequence
  Description of Artificial Sequence Synthetic primer
accctcaaatttccacaaaaca

SEQ ID NO: 7 - DNA, 22 bp, Artificial Sequence
  Description of Artificial Sequence Synthetic primer
ttttttaggttttagggtcggc

SEQ ID NO: 8 - DNA, 19 bp, Artificial Sequence
  Description of Artificial Sequence Synthetic primer
ctcgaatttccgcaaaacg

SEQ ID NO: 9 - DNA, 25 bp, Artificial Sequence
  Description of Artificial Sequence Synthetic primer
gggagtaggttgaggtatttgaagt

SEQ ID NO: 10 - DNA, 22 bp, Artificial Sequence
  Description of Artificial Sequence Synthetic primer
caaaccaaaaaataaacaacca

SEQ ID NO: 11 - DNA, 24 bp, Artificial Sequence
  Description of Artificial Sequence Synthetic primer
ggagtaggttgaggtattcgaagc

SEQ ID NO: 12 - DNA, 22 bp, Artificial Sequence
  Description of Artificial Sequence Synthetic primer
cgaaccaaaaaataaacgaccg

SEQ ID NO: 13 - DNA, 23 bp, Artificial Sequence
  Description of Artificial Sequence Synthetic primer
tggtagtgattgtggttgattat

SEQ ID NO: 14 - DNA, 23 bp, Artificial Sequence
  Description of Artificial Sequence Synthetic primer
cctcacaccaataccacaaaaca

SEQ ID NO: 15 - DNA, 22 bp, Artificial Sequence
  Description of Artificial Sequence Synthetic primer
ggtagcgatcgcggttgattac

SEQ ID NO: 16 - DNA, 22 bp, Artificial Sequence
  Description of Artificial Sequence Synthetic primer
ctcacgccgataccacgaaacg

SEQ ID NO: 17 - DNA, 24 bp, Artificial Sequence
  Description of Artificial Sequence Synthetic primer
ggtattttagatgttttgattttg

SEQ ID NO: 18 - DNA, 23 bp, Artificial Sequence
  Description of Artificial Sequence Synthetic primer
aaaacaacttatccacccaccaa

SEQ ID NO: 19 - DNA, 24 bp, Artificial Sequence
  Description of Artificial Sequence Synthetic primer
ggtattttagacgtttcgatttcg

SEQ ID NO: 20 - DNA, 23 bp, Artificial Sequence
  Description of Artificial Sequence Synthetic primer
gaaacaacttatccacccgccga

SEQ ID NO: 21 - DNA, 20 bp, Artificial Sequence
  Description of Artificial Sequence Synthetic primer
gaaggggagagattggaggt

SEQ ID NO: 22 - DNA, 20 bp, Artificial Sequence
  Description of Artificial Sequence Synthetic primer
tcaccatcaccacaacaaca

SEQ ID NO: 23 - DNA, 20 bp, Artificial Sequence
  Description of Artificial Sequence Synthetic primer
gaaggggagagatcggaggc

SEQ ID NO: 24 - DNA, 20 bp, Artificial Sequence
  Description of Artificial Sequence Synthetic primer
tcaccatcaccacgacgacg

SEQ ID NO: 25 - DNA, 20 bp, Artificial Sequence
  Description of Artificial Sequence Synthetic primer
ggagatgtttttaggtgatt

SEQ ID NO: 26 - DNA, 20 bp, Artificial Sequence
  Description of Artificial Sequence Synthetic primer
acaacacatctcatcttaca

SEQ ID NO: 27 - DNA, 20 bp, Artificial Sequence
  Description of Artificial Sequence Synthetic primer
ggagacgtttttaggcgatc

SEQ ID NO: 28 - DNA, 20 bp, Artificial Sequence
  Description of Artificial Sequence Synthetic primer
acaacacgtctcatcttacg

SEQ ID NO: 29 - DNA, 23 bp, Artificial Sequence
  Description of Artificial Sequence Synthetic primer
tgtttatttttgttttgtaggtg

SEQ ID NO: 30 - DNA, 20 bp, Artificial Sequence
  Description of Artificial Sequence Synthetic primer
aaaactcaaaaccattccaa

SEQ ID NO: 31 - DNA, 22 bp, Artificial Sequence
  Description of Artificial Sequence Synthetic primer
tgtttattttcgtttcgtaggc

SEQ ID NO: 32 - DNA, 20 bp, Artificial Sequence
  Description of Artificial Sequence Synthetic primer
aaaactcaaaaccgttccga

SEQ ID NO: 33 - DNA, 22 bp, Artificial Sequence
  Description of Artificial Sequence Synthetic primer
ttgggattaggtttagttttgg

SEQ ID NO: 34 - DNA, 25 bp, Artificial Sequence
  Description of Artificial Sequence Synthetic primer
ctataaaaccctatcaactcacact

SEQ ID NO: 35 - DNA, 21 bp, Artificial Sequence
  Description of Artificial Sequence Synthetic primer
tcgggattaggtttagtttcg

SEQ ID NO: 36 - DNA, 20 bp, Artificial Sequence
  Description of Artificial Sequence Synthetic primer
aaaccctatcgactcacgct

SEQ ID NO: 37 - DNA, 24 bp, Artificial Sequence
  Description of Artificial Sequence Synthetic primer
gtattttaggaagttgttgtttgt

SEQ ID NO: 38 - DNA, 22 bp, Artificial Sequence
  Description of Artificial Sequence Synthetic primer
ttttctccccaacctcccaaca

SEQ ID NO: 39 - DNA, 24 bp, Artificial Sequence
  Description of Artificial Sequence Synthetic primer
gtattttaggaagtcgttgtttgc

SEQ ID NO: 40 - DNA, 23 bp, Artificial Sequence
  Description of Artificial Sequence Synthetic primer
tttttctccccaacctcccgacg

SEQ ID NO: 41 - DNA, 23 bp, Artificial Sequence
  Description of Artificial Sequence Synthetic primer
ggttaggttttgtgttttgatgt

SEQ ID NO: 42 - DNA, 21 bp, Artificial Sequence
  Description of Artificial Sequence Synthetic primer
cctaactacacactccatcca

SEQ ID NO: 43 - DNA, 23 bp, Artificial Sequence
  Description of Artificial Sequence Synthetic primer
ggttaggttttgtgttttgacgc

SEQ ID NO: 44 - DNA, 21 bp, Artificial Sequence
  Description of Artificial Sequence Synthetic primer
cctaactacgcgctccatccg

SEQ ID NO: 45 - DNA, 22 bp, Artificial Sequence
  Description of Artificial Sequence Synthetic primer
tttttaatttagtggggtttgt

SEQ ID NO: 46 - DNA, 20 bp, Artificial Sequence
  Description of Artificial Sequence Synthetic primer
aaaaacatccaaccaatcca

SEQ ID NO: 47 - DNA, 20 bp, Artificial Sequence
  Description of Artificial Sequence Synthetic primer
tttaatttagcggggttcgc

SEQ ID NO: 48 - DNA, 20 bp, Artificial Sequence
  Description of Artificial Sequence Synthetic primer
aaaaacgtccaaccaatccg

SEQ ID NO: 49 - DNA, 22 bp, Artificial Sequence
  Description of Artificial Sequence Synthetic primer
tgtttgttttgggaagagttgt

SEQ ID NO: 50 - DNA, 22 bp, Artificial Sequence
  Description of Artificial Sequence Synthetic primer
aactccaaaatttcacacccca

SEQ ID NO: 51 - DNA, 22 bp, Artificial Sequence
  Description of Artificial Sequence Synthetic primer
tgttcgtttcgggaagagtcgc

SEQ ID NO: 52 - DNA, 22 bp, Artificial Sequence
  Description of Artificial Sequence Synthetic primer
aactccgaaatttcgcgccccg

SEQ ID NO: 53 - DNA, 18 bp, Artificial Sequence
  Description of Artificial Sequence Synthetic primer
tgtaaaatggattgggtg

SEQ ID NO: 54 - DNA, 18 bp, Artificial Sequence
  Description of Artificial Sequence Synthetic primer
aaaactctcaaaccccac

SEQ ID NO: 55 - DNA, 18 bp, Artificial Sequence
  Description of Artificial Sequence Synthetic primer
ttgtaaaacggattgggc

SEQ ID NO: 56 - DNA, 18 bp, Artificial Sequence
  Description of Artificial Sequence Synthetic primer
aaaactctcgaaccccgc

SEQ ID NO: 57 - DNA, 23 bp, Artificial Sequence
  Description of Artificial Sequence Synthetic primer
gatgtgttgtatagttaattggt

SEQ ID NO: 58 - DNA, 20 bp, Artificial Sequence
  Description of Artificial Sequence Synthetic primer
tccccattaaacaacctcca

SEQ ID NO: 59 - DNA, 21 bp, Artificial Sequence
  Description of Artificial Sequence Synthetic primer
cgcgtcgtatagttaatcggc

SEQ ID NO: 60 - DNA, 20 bp, Artificial Sequence
  Description of Artificial Sequence Synthetic primer
tccccgttaaacgacctccg

SEQ ID NO: 61 - DNA, 24 bp, Artificial Sequence
  Description of Artificial Sequence Synthetic primer
tggggttagatttttgttgttttt

SEQ ID NO: 62 - DNA, 23 bp, Artificial Sequence
  Description of Artificial Sequence Synthetic primer
ataaaatcttacaaccaccatca

SEQ ID NO: 63 - DNA, 24 bp, Artificial Sequence
  Description of Artificial Sequence Synthetic primer
cggggttagattttcgttgttttc

SEQ ID NO: 64 - DNA, 23 bp, Artificial Sequence
  Description of Artificial Sequence Synthetic primer
ataaaatcttacgaccaccgtcg

SEQ ID NO: 65 - DNA, 23 bp, Artificial Sequence
  Description of Artificial Sequence Synthetic primer
gttgttttaatgggagtagggat

SEQ ID NO: 66 - DNA, 23 bp, Artificial Sequence
  Description of Artificial Sequence Synthetic primer
caaaataaaacaaaaacacctca

SEQ ID NO: 67 - DNA, 23 bp, Artificial Sequence
  Description of Artificial Sequence Synthetic primer
gtcgttttaatgggagtagggac

SEQ ID NO: 68 - DNA, 22 bp, Artificial Sequence
  Description of Artificial Sequence Synthetic primer
gaaataaaacgaaaacgcctcg

SEQ ID NO: 69 - DNA, 22 bp, Artificial Sequence
  Description of Artificial Sequence Synthetic primer
gtaggaggatttttggtttgtt

SEQ ID NO: 70 - DNA, 22 bp, Artificial Sequence
  Description of Artificial Sequence Synthetic primer
cctaaattacaacccaacctca

SEQ ID NO: 71 - DNA, 20 bp, Artificial Sequence
  Description of Artificial Sequence Synthetic primer
aggaggatttttggttcgtc

SEQ ID NO: 72 - DNA, 20 bp, Artificial Sequence
  Description of Artificial Sequence Synthetic primer
taaattacgacccaacctcg

SEQ ID NO: 73 - DNA, 29 bp, Artificial Sequence
  Description of Artificial Sequence Synthetic primer
ttttgatgtagatgttttattagggttgt

SEQ ID NO: 74 - DNA, 24 bp, Artificial Sequence
  Description of Artificial Sequence Synthetic primer
accacctcatcataactacccaca

SEQ ID NO: 75 - DNA, 24 bp, Artificial Sequence
  Description of Artificial Sequence Synthetic primer
acgtagacgttttattagggtcgc

SEQ ID NO: 76 - DNA, 20 bp, Artificial Sequence
  Description of Artificial Sequence Synthetic primer
cctcatcgtaactacccgcg

SEQ ID NO: 77 - DNA, 24 bp, Artificial Sequence
  Description of Artificial Sequence Synthetic primer
gattttaggattgttgatatgagt

SEQ ID NO: 78 - DNA, 24 bp, Artificial Sequence
  Description of Artificial Sequence Synthetic primer
aaactacctcctaatctttatcca

SEQ ID NO: 79 - DNA, 24 bp, Artificial Sequence
  Description of Artificial Sequence Synthetic primer
gattttaggattgtcgatatgagc

SEQ ID NO: 80 - DNA, 23 bp, Artificial Sequence
  Description of Artificial Sequence Synthetic primer
aactacctcctaatctttatccg

SEQ ID NO: 81 - DNA, 27 bp, Artificial Sequence
  Description of Artificial Sequence Synthetic primer
ggtgaatttttagttaattagtggtat

SEQ ID NO: 82 - DNA, 25 bp, Artificial Sequence
  Description of Artificial Sequence Synthetic primer
tcacaaatactttacaattccaaca

SEQ ID NO: 83 - DNA, 25 bp, Artificial Sequence
  Description of Artificial Sequence Synthetic primer
tgaatttttagttaattagcggtac

SEQ ID NO: 84 - DNA, 23 bp, Artificial Sequence
  Description of Artificial Sequence Synthetic primer
acaaatactttacaattccgacg

SEQ ID NO: 85 - DNA, 23 bp, Artificial Sequence
  Description of Artificial Sequence Synthetic primer
ttttgtgttttgtaagaattggt

SEQ ID NO: 86 - DNA, 23 bp, Artificial Sequence
  Description of Artificial Sequence Synthetic primer
aacctcccaaaaaaacactatca

SEQ ID NO: 87 - DNA, 23 bp, Artificial Sequence
  Description of Artificial Sequence Synthetic primer
tttcgcgttttgtaagaatcggc

SEQ ID NO: 88 - DNA, 22 bp, Artificial Sequence
  Description of Artificial Sequence Synthetic primer
acctcccgaaaaaacgctatcg

SEQ ID NO: 89 - DNA, 22 bp, Artificial Sequence
  Description of Artificial Sequence Synthetic primer
tattttgttgggtttttatggt

SEQ ID NO: 90 - DNA, 25 bp, Artificial Sequence
  Description of Artificial Sequence Synthetic primer
aactccaaatcaattcacaacatca

SEQ ID NO: 91 - DNA, 22 bp, Artificial Sequence
  Description of Artificial Sequence Synthetic primer
tattttgtcgggtttttacggc

SEQ ID NO: 92 - DNA, 19 bp, Artificial Sequence
  Description of Artificial Sequence Synthetic primer
aaatcgattcgcgacgtcg

SEQ ID NO: 93 - DNA, 25 bp, Artificial Sequence
  Description of Artificial Sequence Synthetic primer
taatggtttttgaggattgagattg

SEQ ID NO: 94 - DNA, 25 bp, Artificial Sequence
  Description of Artificial Sequence Synthetic primer
cacaaactattatcaaccaatcaca

SEQ ID NO: 95 - DNA, 24 bp, Artificial Sequence
  Description of Artificial Sequence Synthetic primer
taatggtttttgaggattgagatc

SEQ ID NO: 96 - DNA, 25 bp, Artificial Sequence
  Description of Artificial Sequence Synthetic primer
cacaaactattatcaaccgatcacg

SEQ ID NO: 97 - DNA, 22 bp, Artificial Sequence
  Description of Artificial Sequence Synthetic primer
tggttgttatttggggtggttg

SEQ ID NO: 98 - DNA, 23 bp, Artificial Sequence
  Description of Artificial Sequence Synthetic primer
ctaaaccacactaaaaacaaaca

SEQ ID NO: 99 - DNA, 21 bp, Artificial Sequence
  Description of Artificial Sequence Synthetic primer
tggttgttatttggggcggtc

SEQ ID NO: 100 - DNA, 23 bp, Artificial Sequence
  Description of Artificial Sequence Synthetic primer
ctaaaccacactaaaaacgaacg

SEQ ID NO: 101 - DNA, 24 bp, Artificial Sequence
  Description of Artificial Sequence Synthetic primer
ggttttggaaattgtatatggtgt

SEQ ID NO: 102 - DNA, 23 bp, Artificial Sequence
  Description of Artificial Sequence Synthetic primer
aaacaacaaatctcaaacctaca

SEQ ID NO: 103 - DNA, 22 bp, Artificial Sequence
  Description of Artificial Sequence Synthetic primer
tttcggaaattgtatacggcgc

SEQ ID NO: 104 - DNA, 22 bp, Artificial Sequence
  Description of Artificial Sequence Synthetic primer
aacaacgaatctcgaacctacg

SEQ ID NO: 105 - DNA, 23 bp, Artificial Sequence
  Description of Artificial Sequence Synthetic primer
tgtttgagtgtatatgagtggat

SEQ ID NO: 106 - DNA, 24 bp, Artificial Sequence
  Description of Artificial Sequence Synthetic primer
tctctcaaatcccacaaacaacca

SEQ ID NO: 107 - DNA, 23 bp, Artificial Sequence
  Description of Artificial Sequence Synthetic primer
tgttcgagtgtatatgagtggac

SEQ ID NO: 108 - DNA, 24 bp, Artificial Sequence
  Description of Artificial Sequence Synthetic primer
tctctcgaatcccacaaacgaccg

SEQ ID NO: 109 - DNA, 21 bp, Artificial Sequence
  Description of Artificial Sequence Synthetic primer
aggtttagttagttttagttg

SEQ ID NO: 110 - DNA, 21 bp, Artificial Sequence
  Description of Artificial Sequence Synthetic primer
ccactaaataccctaacaaca

SEQ ID NO: 111 - DNA, 21 bp, Artificial Sequence
  Description of Artificial Sequence Synthetic primer
aggtttagttagttttagtcg

SEQ ID NO: 112 - DNA, 21 bp, Artificial Sequence
  Description of Artificial Sequence Synthetic primer
ccactaaataccctaacaacg

SEQ ID NO: 113 - DNA, 23 bp, Artificial Sequence
  Description of Artificial Sequence Synthetic primer
gtttgagggtgggtggtagttgt

SEQ ID NO: 114 - DNA, 23 bp, Artificial Sequence
  Description of Artificial Sequence Synthetic primer
actaccccaaaaaatctaaatca

SEQ ID NO: 115 - DNA, 23 bp, Artificial Sequence
  Description of Artificial Sequence Synthetic primer
gtttgagggcgggtggtagtcgc

SEQ ID NO: 116 - DNA, 23 bp, Artificial Sequence
  Description of Artificial Sequence Synthetic primer
actaccccgaaaaatctaaatcg

SEQ ID NO: 117 - DNA, 24 bp, Artificial Sequence
  Description of Artificial Sequence Synthetic primer
ggtaggagttattaggagagtatt

SEQ ID NO: 118 - DNA, 25 bp, Artificial Sequence
  Description of Artificial Sequence Synthetic primer
aatacaaacactcaccacctaacca

SEQ ID NO: 119 - DNA, 24 bp, Artificial Sequence
  Description of Artificial Sequence Synthetic primer
ggtaggagttattaggagagtatc

SEQ ID NO: 120 - DNA, 25 bp, Artificial Sequence
  Description of Artificial Sequence Synthetic primer
aatacaaacgctcaccgcctaaccg

SEQ ID NO: 121 - DNA, 23 bp, Artificial Sequence
  Description of Artificial Sequence Synthetic primer
ttttttttaaggattttgtgtgt

SEQ ID NO: 122 - DNA, 22 bp, Artificial Sequence
  Description of Artificial Sequence Synthetic primer
actaaaaatacacattccacca

SEQ ID NO: 123 - DNA, 22 bp, Artificial Sequence
  Description of Artificial Sequence Synthetic primer
tttttttaaggatttcgcgcgc

SEQ ID NO: 124 - DNA, 25 bp, Artificial Sequence
  Description of Artificial Sequence Synthetic primer
ccaactaaaaatacacgttccaccg

SEQ ID NO: 125 - DNA, 19 bp, Artificial Sequence
  Description of Artificial Sequence Synthetic primer
gtggttaggaatggaagtg

SEQ ID NO: 126 - DNA, 19 bp, Artificial Sequence
  Description of Artificial Sequence Synthetic primer
aacccaacctccaccatca

SEQ ID NO: 127 - DNA, 19 bp, Artificial Sequence
  Description of Artificial Sequence Synthetic primer
ggcggttaggaacggaagc

SEQ ID NO: 128 - DNA, 19 bp, Artificial Sequence
  Description of Artificial Sequence Synthetic primer
aacccgacctccgccatcg

SEQ ID NO: 129 - DNA, 25 bp, Artificial Sequence
  Description of Artificial Sequence Synthetic primer
gaattttgtgatttatgtgatgatg

SEQ ID NO: 130 - DNA, 24 bp, Artificial Sequence
  Description of Artificial Sequence Synthetic primer
ctctaaacaaacacaaacaataca

SEQ ID NO: 131 - DNA, 24 bp, Artificial Sequence
  Description of Artificial Sequence Synthetic primer
gaatttcgtgatttacgtgacgac

SEQ ID NO: 132 - DNA, 24 bp, Artificial Sequence
  Description of Artificial Sequence Synthetic primer
ctctaaacaaacacaaacgatacg

SEQ ID NO: 133 - DNA, 25 bp, Artificial Sequence
  Description of Artificial Sequence Synthetic primer
gaatattttattttttggttttgtg

SEQ ID NO: 134 - DNA, 21 bp, Artificial Sequence
  Description of Artificial Sequence Synthetic primer
aaaaaacctccaacatattca

SEQ ID NO: 135 - DNA, 18 bp, Artificial Sequence
  Description of Artificial Sequence Synthetic primer
ttatttttcggttttgcg

SEQ ID NO: 136 - DNA, 21 bp, Artificial Sequence
  Description of Artificial Sequence Synthetic primer
aaaaaacctccaacatattcg

SEQ ID NO: 137 - DNA, 24 bp, Artificial Sequence
  Description of Artificial Sequence Synthetic primer
gtaatagtaagtgtatggagttgt

SEQ ID NO: 138 - DNA, 22 bp, Artificial Sequence
  Description of Artificial Sequence Synthetic primer
cctacaaaaatctaaataacca

SEQ ID NO: 139 - DNA, 24 bp, Artificial Sequence
  Description of Artificial Sequence Synthetic primer
gtaatagtaagcgtatggagtcgc

SEQ ID NO: 140 - DNA, 22 bp, Artificial Sequence
  Description of Artificial Sequence Synthetic primer
cctacgaaaatctaaataaccg

SEQ ID NO: 141 - DNA, 22 bp, Artificial Sequence
  Description of Artificial Sequence Synthetic primer
ggtagttgtgttttgtgtaggt

SEQ ID NO: 142 - DNA, 20 bp, Artificial Sequence
  Description of Artificial Sequence Synthetic primer
cacataaccaattttccaca

SEQ ID NO: 143 - DNA, 22 bp, Artificial Sequence
  Description of Artificial Sequence Synthetic primer
ggtagttgtgttttgtgtaggc

SEQ ID NO: 144 - DNA, 20 bp, Artificial Sequence
  Description of Artificial Sequence Synthetic primer
cacgtaaccgattttccacg

SEQ ID NO: 145 - DNA, 20 bp, Artificial Sequence
  Description of Artificial Sequence Synthetic primer
agggtagggtttgagttgtt

SEQ ID NO: 146 - DNA, 20 bp, Artificial Sequence
  Description of Artificial Sequence Synthetic primer
aatacctcacaataccccca

SEQ ID NO: 147 - DNA, 20 bp, Artificial Sequence
  Description of Artificial Sequence Synthetic primer
agggtagggtttgagtcgtc

SEQ ID NO: 148 - DNA, 20 bp, Artificial Sequence
  Description of Artificial Sequence Synthetic primer
aatacctcgcaatacccccg

SEQ ID NO: 149 - DNA, 24 bp, Artificial Sequence
  Description of Artificial Sequence Synthetic primer
ggagagattagagatgattgttgt

SEQ ID NO: 150 - DNA, 23 bp, Artificial Sequence
  Description of Artificial Sequence Synthetic primer
cataaactcttatccccaataca

SEQ ID NO: 151 - DNA, 24 bp, Artificial Sequence
  Description of Artificial Sequence Synthetic primer
ggagagattagagatgatcgtcgc

SEQ ID NO: 152 - DNA, 23 bp, Artificial Sequence
  Description of Artificial Sequence Synthetic primer
cgtaaactcttatccccaatacg

SEQ ID NO: 153 - DNA, 22 bp, Artificial Sequence
  Description of Artificial Sequence Synthetic primer
tggtttttggggtaatttttgt

SEQ ID NO: 154 - DNA, 22 bp, Artificial Sequence
  Description of Artificial Sequence Synthetic primer
caaatctacaacctatttccca

SEQ ID NO: 155 - DNA, 23 bp, Artificial Sequence
  Description of Artificial Sequence Synthetic primer
ttttatacgattcggaattcgac

SEQ ID NO: 156 - DNA, 22 bp, Artificial Sequence
  Description of Artificial Sequence Synthetic primer
caaatctacgacctatttcccg

SEQ ID NO: 157 - DNA, 20 bp, Artificial Sequence
  Description of Artificial Sequence Synthetic primer
gttaggttgttgtttagggt

SEQ ID NO: 158 - DNA, 20 bp, Artificial Sequence
  Description of Artificial Sequence Synthetic primer
ccaaaaaaatcacaaaccca

SEQ ID NO: 159 - DNA, 22 bp, Artificial Sequence
  Description of Artificial Sequence Synthetic primer
gcgtttgttttttttcgtcgac

SEQ ID NO: 160 - DNA, 23 bp, Artificial Sequence
  Description of Artificial Sequence Synthetic primer
aaatcacgaacccgaatataacg

SEQ ID NO: 161 - DNA, 22 bp, Artificial Sequence
  Description of Artificial Sequence Synthetic primer
aatacacacaaatgtctctccc

SEQ ID NO: 162 - DNA, 20 bp, Artificial Sequence
  Description of Artificial Sequence Synthetic primer
cctttttttcagtggtatgc

SEQ ID NO: 163 - DNA, 21 bp, Artificial Sequence
  Description of Artificial Sequence Synthetic primer
attccatactgtagggagtgt

SEQ ID NO: 164 - DNA, 17 bp, Artificial Sequence
  Description of Artificial Sequence Synthetic primer
gcaaatgccatgctgta

SEQ ID NO: 165 - DNA, 20 bp, Artificial Sequence
  Description of Artificial Sequence Synthetic primer
cttctcactgcatatgactc

SEQ ID NO: 166 - DNA, 16 bp, Artificial Sequence
  Description of Artificial Sequence Synthetic primer
ggctccccagggtaag

SEQ ID NO: 167 - DNA, 20 bp, Artificial Sequence
  Description of Artificial Sequence Synthetic primer
gatgaaactgtgatagcacc

SEQ ID NO: 168 - DNA, 21 bp, Artificial Sequence
  Description of Artificial Sequence Synthetic primer
ctgccagtaatgtaaatctcc

SEQ ID NO: 169 - DNA, 18 bp, Artificial Sequence
  Description of Artificial Sequence Synthetic primer
gtcctgcaagactcattg

SEQ ID NO: 170 - DNA, 20 bp, Artificial Sequence
  Description of Artificial Sequence Synthetic primer
ttgctaggatggttgttttc

SEQ ID NO: 171 - DNA, 19 bp, Artificial Sequence
  Description of Artificial Sequence Synthetic primer
tctgaaaatgcccttgacc

SEQ ID NO: 172 - DNA, 20 bp, Artificial Sequence
  Description of Artificial Sequence Synthetic primer
catcattactgctgggatgc

SEQ ID NO: 173 - DNA, 19 bp, Artificial Sequence
  Description of Artificial Sequence Synthetic primer
gtcaagagggctgattctg

SEQ ID NO: 174 - DNA, 19 bp, Artificial Sequence
  Description of Artificial Sequence Synthetic primer
acctgtctgaacttgcgtg

SEQ ID NO: 175 - DNA, 20 bp, Artificial Sequence
  Description of Artificial Sequence Synthetic primer
gcagttcaaagatgaaagtg

SEQ ID NO: 176 - DNA, 20 bp, Artificial Sequence
  Description of Artificial Sequence Synthetic primer
cattcctagatgggtaaagc

SEQ ID NO: 177 - DNA, 20 bp, Artificial Sequence
  Description of Artificial Sequence Synthetic primer
cacataacttcccttgctgg

SEQ ID NO: 178 - DNA, 20 bp, Artificial Sequence
  Description of Artificial Sequence Synthetic primer
actgttgtcaaatcaggctc

SEQ ID NO: 179 - DNA, 20 bp, Artificial Sequence
  Description of Artificial Sequence Synthetic primer
ggtaaatagggaaaatgaca

SEQ ID NO: 180 - DNA, 20 bp, Artificial Sequence
  Description of Artificial Sequence Synthetic primer
ttaggatgctgacttcacca

SEQ ID NO: 181 - DNA, 20 bp, Artificial Sequence
  Description of Artificial Sequence Synthetic primer
ctactaccagcagcattctc

SEQ ID NO: 182 - DNA, 20 bp, Artificial Sequence
  Description of Artificial Sequence Synthetic primer
atctgcagtgtgaggcaatg

SEQ ID NO: 183 - DNA, 23 bp, Artificial Sequence
  Description of Artificial Sequence Synthetic primer
actcactctagtgataaatcggg

SEQ ID NO: 184 - DNA, 25 bp, Artificial Sequence
  Description of Artificial Sequence Synthetic primer
agcagataagacagtattactagtt

SEQ ID NO: 185 - DNA, 20 bp, Artificial Sequence
  Description of Artificial Sequence Synthetic primer
gggatgaagtgtggataaac

SEQ ID NO: 186 - DNA, 20 bp, Artificial Sequence
  Description of Artificial Sequence Synthetic primer
taggatggcagtgctcttag

SEQ ID NO: 187 - DNA, 24 bp, Artificial Sequence
  Description of Artificial Sequence Synthetic primer
atttggtttccatagaatctgaga

SEQ ID NO: 188 - DNA, 21 bp, Artificial Sequence
  Description of Artificial Sequence Synthetic primer
ttacacccaccagattaagcg

SEQ ID NO: 189 - DNA, 24 bp, Artificial Sequence
  Description of Artificial Sequence Synthetic primer
ttaattgatctgggctggagaacc

SEQ ID NO: 190 - DNA, 24 bp, Artificial Sequence
  Description of Artificial Sequence Synthetic primer
cagagcaaggtcctgtctgaaaat

SEQ ID NO: 191 - DNA, 20 bp, Artificial Sequence
  Description of Artificial Sequence Synthetic primer
tgccactgtcttgaaaatcc

SEQ ID NO: 192 - DNA, 20 bp, Artificial Sequence
  Description of Artificial Sequence Synthetic primer
tatggcccagcaatgtgtat

SEQ ID NO: 193 - DNA, 20 bp, Artificial Sequence
  Description of Artificial Sequence Synthetic primer
agctcaaaagcgaaggtgat

SEQ ID NO: 194 - DNA, 20 bp, Artificial Sequence
  Description of Artificial Sequence Synthetic primer
ggcaacaaagtgagatcctg

SEQ ID NO: 195 - DNA, 21 bp, Artificial Sequence
  Description of Artificial Sequence Synthetic primer
cgtttggaaattgtcattacc

SEQ ID NO: 196 - DNA, 19 bp, Artificial Sequence
  Description of Artificial Sequence Synthetic primer
tcgctcagaaacaaaccaa

SEQ ID NO: 197 - DNA, 19 bp, Artificial Sequence
  Description of Artificial Sequence Synthetic primer
aggattgtaatttccttgc

SEQ ID NO: 198 - DNA, 19 bp, Artificial Sequence
  Description of Artificial Sequence Synthetic primer
gggactttttgaaggtttg

SEQ ID NO: 199 - DNA, 20 bp, Artificial Sequence
  Description of Artificial Sequence Synthetic primer
gatttgtcctcatgcagtgt

SEQ ID NO: 200 - DNA, 23 bp, Artificial Sequence
  Description of Artificial Sequence Synthetic primer
acatgttatgtttgagaggtctg

SEQ ID NO: 201 - DNA, 20 bp, Artificial Sequence
  Description of Artificial Sequence Synthetic primer
agcaaggcaagccacatttc

SEQ ID NO: 202 - DNA, 24 bp, Artificial Sequence
  Description of Artificial Sequence Synthetic primer
tggggatgcccagataactatatc

SEQ ID NO: 203 - DNA, 25 bp, Artificial Sequence
  Description of Artificial Sequence Synthetic primer
gcatttcacaggaaataatctaagg

SEQ ID NO: 204 - DNA, 18 bp, Artificial Sequence
  Description of Artificial Sequence Synthetic primer
cctctctgcatgccccag

SEQ ID NO: 205 - DNA, 22 bp, Artificial Sequence
  Description of Artificial Sequence Synthetic primer
aggtgtagtccttctggaattt

SEQ ID NO: 206 - DNA, 25 bp, Artificial Sequence
  Description of Artificial Sequence Synthetic primer
gatgtgactgctgttaaaactagag

SEQ ID NO: 207 - DNA, 23 bp, Artificial Sequence
  Description of Artificial Sequence Synthetic primer
acacattcataccatagcagagg

SEQ ID NO: 208 - DNA, 20 bp, Artificial Sequence
  Description of Artificial Sequence Synthetic primer
ggggaaagcattcagacttt

SEQ ID NO: 209 - DNA, 16 bp, Artificial Sequence
  Description of Artificial Sequence Synthetic primer
agcaacctcaacaggg

SEQ ID NO: 210 - DNA, 18 bp, Artificial Sequence
  Description of Artificial Sequence Synthetic primer
aatcatccagaaaggcca

SEQ ID NO: 211 - DNA, 18 bp, Artificial Sequence
  Description of Artificial Sequence Synthetic primer
ggcctgaaaggtatcctc

SEQ ID NO: 212 - DNA, 18 bp, Artificial Sequence
  Description of Artificial Sequence Synthetic primer
tcccaccataagcacaag

SEQ ID NO: 213 - DNA, 25 bp, Artificial Sequence
  Description of Artificial Sequence Synthetic primer
cctggcctgttagtttttattgtta

SEQ ID NO: 214 - DNA, 22 bp, Artificial Sequence
  Description of Artificial Sequence Synthetic primer
cccagtcttgggtatgttttta

SEQ ID NO: 215 - DNA, 24 bp, Artificial Sequence
  Description of Artificial Sequence Synthetic primer
ccctgtcttctacttcctgtatgc

SEQ ID NO: 216 - DNA, 20 bp, Artificial Sequence
  Description of Artificial Sequence Synthetic primer
cggttctcaaccaggagaaa

SEQ ID NO: 217 - DNA, 20 bp, Artificial Sequence
  Description of Artificial Sequence Synthetic primer
tgattgtatgcatgcctgtg

SEQ ID NO: 218 - DNA, 20 bp, Artificial Sequence
  Description of Artificial Sequence Synthetic primer
tagagtgcagtgtccaaacg

SEQ ID NO: 219 - DNA, 20 bp, Artificial Sequence
  Description of Artificial Sequence Synthetic primer
tataacttgtgtgagcacag

SEQ ID NO: 220 - DNA, 20 bp, Artificial Sequence
  Description of Artificial Sequence Synthetic primer
ccacagacatcagagtcctt

SEQ ID NO: 221 - DNA, 24 bp, Artificial Sequence
  Description of Artificial Sequence Synthetic primer
agggatactattcagcccgaggtg

SEQ ID NO: 222 - DNA, 22 bp, Artificial Sequence
  Description of Artificial Sequence Synthetic primer
actgccactccttgccccattc

SEQ ID NO: 223 - DNA, 25 bp, Artificial Sequence
  Description of Artificial Sequence Synthetic primer
cagaaccacaaaatgtcttgcattc

SEQ ID NO: 224 - DNA, 22 bp, Artificial Sequence
  Description of Artificial Sequence Synthetic primer
ggccagacagaccaggctctgc

SEQ ID NO: 225 - DNA, 20 bp, Artificial Sequence
  Description of Artificial Sequence Synthetic primer
caatggaaccaaatgtggtc

SEQ ID NO: 226 - DNA, 20 bp, Artificial Sequence
  Description of Artificial Sequence Synthetic primer
agtccgataatgccaggatg

SEQ ID NO: 227 - DNA, 24 bp, Artificial Sequence
  Description of Artificial Sequence Synthetic primer
cctaattacacaatacttttgggg

SEQ ID NO: 228 - DNA, 24 bp, Artificial Sequence
  Description of Artificial Sequence Synthetic primer
taatataagactaacaaacaaatg

SEQ ID NO: 229 - DNA, 23 bp, Artificial Sequence
  Description of Artificial Sequence Synthetic primer
ttactgagctgtaattccatgat

SEQ ID NO: 230 - DNA, 23 bp, Artificial Sequence
  Description of Artificial Sequence Synthetic primer
ctcttaccttgctggtgagattg

SEQ ID NO: 231 - DNA, 22 bp, Artificial Sequence
  Description of Artificial Sequence Synthetic primer
aaggagtaacttgggttccatc

SEQ ID NO: 232 - DNA, 21 bp, Artificial Sequence
  Description of Artificial Sequence Synthetic primer
cctgcacttgatgagataggc

SEQ ID NO: 233 - DNA, 20 bp, Artificial Sequence
  Description of Artificial Sequence Synthetic primer
ttcagggtcttttgaagagg

SEQ ID NO: 234 - DNA, 20 bp, Artificial Sequence
  Description of Artificial Sequence Synthetic primer
agaaggcattaaattttgca

SEQ ID NO: 235 - DNA, 19 bp, Artificial Sequence
  Description of Artificial Sequence Synthetic primer
tggggtgtttaccagcatc

SEQ ID NO: 236 - DNA, 20 bp, Artificial Sequence
  Description of Artificial Sequence Synthetic primer
tggctttcaatgtcagaagg

SEQ ID NO: 237 - DNA, 17 bp, Artificial Sequence
  Description of Artificial Sequence Synthetic primer
aaggctganctctaccg

SEQ ID NO: 238 - DNA, 25 bp, Artificial Sequence
  Description of Artificial Sequence Synthetic primer
ggaatgtcaagaagtacctaccata

SEQ ID NO: 239 - DNA, 16 bp, Artificial Sequence
  Description of Artificial Sequence Synthetic primer
gctcccggctggtttt

SEQ ID NO: 240 - DNA, 20 bp, Artificial Sequence
  Description of Artificial Sequence Synthetic primer
gcaggaaatcgcaggaactt

SEQ ID NO: 241 - protein, 4 aa, Artificial Sequence
  Description of Artificial Sequence Synthetic peptide
DEAD

(19) United States
(12) Patent Application Publication
(10) Pub. No.: US 2010/0028875 A1
(43) Pub. Date: Feb. 4, 2010

(54) METHOD FOR DIAGNOSING CANCER BY DETECTING THE METHYLATION OF TRANSITIONAL ZONES

(76) Inventors: Mun-Gan Rhyu, Seoul (KR); Seung-Jin Hong, Seoul (KR); Young-Ho Kim, Kyunggi-Do (KR); Yu-Chae Jung, Seoul (KR)

Correspondence Address: North Star Intellectual Property Law, PC, P.O. Box 34688, Washington DC, DC, 20043, US

(21) Appl. No.: 12/307,212

(22) PCT Filed: Aug. 19, 2006
(86) PCT No.: PCT/KR06/03267

§ 371 Date: Jun. 2, 2009

(30) Foreign Application Priority Data
Jul. 5, 2006 (KR) 10-2006-0063146

(51) Int. Cl. C12Q 1/68; C07H 21/04

(52) U.S. Cl. 435/6; 536/23.1; 536/24.33

(57) ABSTRACT

The present invention relates to a method for diagnosing cancer and predicting metastasis or prognosis by measuring the methylation of transitional zones and a primer for detecting the methylation. According to the present invention, a novel transitional zone is understood and a primer for detecting the methylation of the zone is provided, indicating that the present invention contributes to increase accuracy and liability of cancer prediction by measuring the methylation of transitional zones and chromosomal loss at the same time.

### TECHNICAL FIELD

The present invention relates to a method for diagnosing cancer by detecting the methylation of transitional zones, confirming the metastasis and prognosis and a primer for detecting methylation.

### BACKGROUND ART

Cancer has been acknowledged as a genetic disorder caused by mutation of a gene. The amino acid sequence and the functions of a protein are determined by the nucleotide sequence of DNA. However, the expression of a protein is affected by DNA methylation. That is, the functions and expression of a specific gene depend on the nucleotide sequence and DNA methylation. In tumor tissues, those genetic and epigenetic changes are generally observed. Therefore, by detecting such genetic and epigenetic changes in tumor tissues a cause of a specific cancer can be explained, providing advantageous information for studies on the prevention and treatment of cancer.

The aspects of DNA methylation in tumor tissues are as follows:

1) Both DNA methylation and demethylation are observed in tumor tissues,

2) DNA methylation is observed in CpG islands adjacent genes, while DNA demethylation is observed in repeated sequences, and

3) DNA methylation and demethylation play an independent role in the development and progress of cancer.

The details of the involvement of DNA methylation in cancer development and progress have not been clearly understood, yet. However, the various aspects of DNA methylation involved in cell growth, differentiation and aging have been found out, making DNA methylation a major target of study to understand various malignant phenotypes caused by genetic instability.

Human genome is differentiated into each tissue by epigenetic reprogramming in the early stage of development. The epigenetic structure is mostly established in the early stage of embryogenesis, which is largely divided into two parts; one is maintained all through the life-time and the other is the transitional zone which is variable according to the cell differentiation and repeated sequence of RNA. Retroelement, which takes more than 40% of human genome, is a repeated sequence originated from endogenous retrovirus-like genetic element. The retroelement induces methylation of itself and at the same time causes methylation of neighboring DNA, indicating that the retroelement plays a crucial role in DNA methylation in the whole genome.

Chromosomal loss can be detected by a simple repeated sequence marker, reflecting the dosage reduction of genome. The dosage reduction brings dosage compensation mechanism in action to keep the dosage of genome in each individual. Thus, chromosomal loss induces demethylation of nucleic acid to compensate the dosage reduction, which seems how DNA methylation is involved in tumor progress. DNA demethylation shows similar gene expression pattern to androgenic program represented by the epigenic activation for cell invasion and transition to induce placentation in the early stage of gestation and the degeneration and loss with parturition.

Therefore, the present inventors investigated DNA methylation in relation to chromosomal loss in tumor tissues, and further confirmed that DNA methylation can play an important role in diagnosing cancer based on the findings by the inventors that DNA methylation is more actively induced in transitional zones in between CPG island and its' neighboring retroelement rather than in CpG island.

1. Balmain A, Gray J, Ponder B. The genetics and genomics of cancer. Nat Genet 2003; 33 S:238-24

2. Hong S J, Choi S W, Lee K H, Lee S, Min K O, Rhyu M G. Preoperative genetic diagnosis of gastric carcinoma based on chromosomal loss and microsatellite instability. Int J Cancer. 2005 Jan. 10; 113(2):249-58.

3. Kim K M, Kwon M S, Hong S J, Min K O, Seo E J, Lee K Y et al. Genetic classification of intestinal-type and diffuse-type gastric cancers based on chromosomal loss and microsatellite instability. Virchows Arch 2003; 443(4):491-50

4. Choi S W, Lee K J, Bae Y A, Min K O, Kwon M S, Kim K M et al. Genetic classification of colorectal cancer based on chromosomal loss and microsatellite instability predicts survival. Clin Cancer Res 2002; 8(7):2311-2322.

### DISCLOSURE

### Technical Problem

It is an object of the present invention to provide a method for diagnosing cancer which enables the prediction of exact level of cancer metastasis and prognosis by examining tumor tissues with endoscope before surgical operation.

### Technical Solution

To achieve the above object, the present invention provides a method for diagnosing cancer and predicting metastasis and prognosis of cancer by investigating DNA methylation of transitional zones.

The present invention also provides a primer for investigating DNA methylation of transitional zones.

The present invention further provides a simple repeated sequence marker group capable of measuring loss of heterozygosity (LOH) and chromosome instability.

Hereinafter, the present invention is described in detail.

The present invention provides a method for diagnosing cancer and predicting metastasis or prognosis by measuring the methylation of transitional zones.

The newly found transitional zone is a variable region formed in between CpG island and its' neighboring retroelement located in the transcription regulating region of the upper stream of a gene, and methylation therein depends on the transcription density of the repeated sequence. In addition to the transcription density dependent methylation, this region exhibits different levels of variability and the big difference in patterns between normal and tumor tissues, indicating that this zone can be utilized for diagnosing cancer.

The following standards are considered for distinguishing genes involved in cancer progress: 1) the distance between a gene and a retroelement; 2) the types of neighboring retroelements; 3) the retroelement density; 4) the distance between genes; 5) relation between CpG island and a retroelement; and 6) the retroelement density in the inside of nucleus. Considering the above standards, 40 epigenes have been identified in relation to diagnosis of cancer, which are divided according to the amount of nucleotide sequence of CpG island into two groups: one includes markers such as RABGEF1, STAG and CHGB prepared in nucleotide sequence rich CPG island and the other group includes TNFRSF14, SERPINB5, ANGPTL7, TFF2, BGLAP, MSLN, DDX53 and MAGEA2 prepared in nucleotide sequence lacking CpG island. They can also be grouped according to the distance between nucleotide of CpG island and its' neighboring retroelement, which are VDR, ST14, CDKN2A, MYBPC2, RUNX3, RUNX2, MLH1, PTEN, etc, prepared in two or more regions including proximal and distal.

If L1 or LTR is more dominant in the upper stream than Alu and if CpG island nucleotide sequence is lacking in the starting point of a gene, the methylation of the transitional zone will be more efficient target for diagnosing cancer and predicting the progress thereof. If CpG island nucleotide sequence is abundant in the starting point of a gene, markers prepared in between a retroelement and CpG island will be highly effective for the prediction.

Methylation can be measured by the conventional methods described in Korean Patent Publication No. 2004-001575, Korean Patent Publication No. 2006-0026595 and Korean Patent Publication No. 2003-0069752.

The present invention also provides a primer for detecting the methylation of the transitional zone.

The primer can be effectively used for measuring the methylation level of the transitional zone, which can be a set of a forward primer and a reverse primer selected from a group consisting of those sequences listed in Table 1. This primer set can be applied to electrophoresis and microarray chip.

TABLE 1

| | | | | |
|---|---|---|---|---|
| CpG | | | Amplicon | |
| nucleotide | Forward | Reverse | size | Tm |
| sequence | (5′to 3′) | (5′to 3′) | (bp) | (° C.) |

| | | | | | |
|---|---|---|---|---|---|
| RABGEF1, | U | AAGTTGGAAGT | CAAAATAAAAT | 131 | 58 |
| −0.2 kb | | AGGGATTGAGT | ACCACCCTAACA | | |
| | M | GTCGGAAGTA | GAAATAAAATA | 128 | 58 |
| | | GGGATTGAGC | CCGCCCTAACG | | |
| STAG1, | U | TTTTTAGGTTT | ACCCTCAAATT | 96 | 58 |
| −0.4 kb | | TAGGGTTGGT | TCCACAAAACA | | |
| | M | TTTTTTAGGTT | CTCGAATTTC | 94 | 58 |
| | | TTAGGGTCGGC | CGCAAAACG | | |
| CHGB, | U | GGGAGTAGGTTG | CAAACCAAAAA | 92 | 58 |
| −0.3 kb | | AGGTATTTGAAGT | ATAAACAACCA | | |
| | M | GGAGTAGGTTG | CGAACCAAAAA | 92 | 58 |
| | | AGGTATTCGAAGC | ATAAACGACCG | | |
| VDR, | U | TGGTAGTGATT | CCTCACACCAA | 130 | 58 |
| −0.7 kb | | GTGGTTGATTAT | TACCACAAAACA | | |
| | M | GGTAGCGATCG | CTCACGCCGAT | 128 | 58 |
| | | CGGTTGATTAC | ACCACGAAACG | | |
| +1.0 kb | U | GGTATTTTAGAT | AAAACAACTTA | 102 | 58 |
| | | GTTTTGATTTTG | TCCACCCACCAA | | |
| | M | GGTATTTTAGAC | GAAACAACTTA | 102 | 58 |
| | | GTTTCGATTTCG | TCCACCCGCCGA | | |
| ST14, | U | GAAGGGGAGA | TCACCATCAC | 136 | 58 |
| −0.3 kb | | GATTGGAGGT | CACAACAACA | | |
| | M | GAAGGGGAGA | TCACCATCAC | 136 | 58 |
| | | GATCGGAGGC | CACGACGACG | | |
| −0.8 kb | U | GGAGATGTTT | ACAACACATC | 106 | 58 |
| | | TTAGGTGATT | TCATCTTACA | | |
| | M | GGAGACGTTT | ACAACACGTC | 106 | 58 |
| | | TTAGGCGATC | TCATCTTACG | | |
| CDKN2A, | U | TGTTTATTTTTG | AAAACTCAAA | 129 | 56 |
| −0.1 kb | | TTTTGTAGGTG | ACCATTCCAA | | |
| | M | TGTTTATTTTC | AAAACTCAAA | 129 | 58 |
| | | GTTTCGTAGGC | ACCGTTCCGA | | |
| −1.5 kb | U | TTGGGATTAGG | CTATAAAACCCT | 130 | 58 |
| | | TTTAGTTTTGG | ATCAACTCACACT | | |
| | M | TCGGGATTAGG | AAACCCTATC | 125 | 60 |
| | | TTTAGTTTCG | GACTCACGCT | | |
| +0.8 kb | U | GTATTTTAGGAA | TTTTCTCCCCA | 101 | 58 |
| | | GTTGTTGTTTGT | ACCTCCCAACA | | |
| | M | GTATTTTAGGAA | TTTTTCTCCCCA | 102 | 60 |
| | | GTCGTTGTTTGC | ACCTCCCGACG | | |
| PPARG, | U | GGTTAGGTTTT | CCTAACTACAC | 111 | 58 |
| −0.2 kb | | GTGTTTTGATGT | ACTCCATCCA | | |
| | M | GGTTAGGTTTT | CCTAACTACGC | 111 | 58 |
| | | GTGTTTTGACGC | GCTCCATCCG | | |
| MYBPC2, | U | TTTTTAATTTA | AAAAACATCC | 96 | 58 |
| −1.2 kb | | GTGGGGTTTGT | AACCAATCCA | | |
| | M | TTTAATTTAG | AAAAACGTCC | 94 | 60 |
| | | CGGGGTTCGC | AACCAATCCG | | |
| −0.7 kb | U | TGTTTGTTTTG | AACTCCAAAAT | 125 | 58 |
| | | GGAAGAGTTGT | TTCACACCCCA | | |
| | M | TGTTCGTTTCG | AACTCCGAAAT | 125 | 60 |
| | | GGAAGAGTCGC | TTCGCGCCCCG | | |
| RB1, | U | TGTAAAATG | AAAACTCTC | 116 | 58 |
| −0.4 kb | | GATTGGGTG | AAACCCCAC | | |
| | M | TTGTAAAAC | AAAACTCTC | 116 | 58 |
| | | GGATTGGGC | GAACCCCGC | | |
| RUNX3, | U | GATGTGTTGTAT | TCCCCATTAA | 97 | 56 |
| −0.5 kb | | AGTTAATTGGT | ACAACCTCCA | | |
| | M | CGCGTCGTAT | TCCCCGTTAA | 95 | 58 |
| | | AGTTAATCGGC | ACGACCTCCG | | |
| 1.7 kb | U | TGGGGTTAGATT | ATAAAATCTTAC | 107 | 56 |
| | | TTTGTTGTTTTT | AACCACCATCA | | |
| | M | CGGGGTTAGATT | ATAAAATCTTAC | 107 | 58 |
| | | TTCGTTGTTTTC | GACCACCGTCG | | |
| +1.0 kb | U | GTTGTTTTAATG | CAAAATAAAACA | 147 | 59 |
| | | GGAGTAGGGAT | AAAACACCTCA | | |
| | M | GTCGTTTTAATG | GAAATAAAACG | 147 | 59 |
| | | GGAGTAGGGAC | AAAACGCCTCG | | |
| PAX5, | U | GTAGGAGGATT | CCTAAATTACA | 115 | 59 |
| −1.0 kb | | TTTGGTTTGTT | ACCCAACCTCA | | |
| | M | AGGAGGATTT | TAAATTACGA | 111 | 59 |
| | | TTGGTTCGTC | CCCAACCTCG | | |
| MLH1, | U | TTTTGATGTAGATG | ACCACCTCATCA | 121 | 58 |
| −0.6 kb | | TTTTATTAGGGTTG | TAACTACCCACA | | |
| | | T | | | |
| | M | ACGTAGACGTTT | CCTCATCGTA | 115 | 58 |
| | | TATTAGGGTCGC | ACTACCCGCG | | |
| −1.0 kb | U | GATTTTAGGATT | AAACTACCTCCT | 126 | 58 |
| | | GTTGATATGAGT | AATCTTTATCCA | | |
| | M | GATTTTAGGATT | AACTACCTCCTA | 125 | 58 |
| | | GTCGATATGAGC | ATCTTTATCCG | | |
| CDH1, | U | GGTGAATTTTTAG | TCACAAATACTT | 108 | 56 |
| 0 kb | | TTAATTAGTGGTAT | TACAATTCCAACA | | |
| | M | TGAATTTTTAGT | ACAAATACTTTA | 104 | 58 |
| | | TAATTAGCGGTAC | CAATTCCGACG | | |
| PTEN, | U | TTTTGTGTTTT | AACCTCCCAAAA | 124 | 58 |
| −1.4 kb | | GTAAGAATTGGT | AAACACTATCA | | |
| | M | TTTCGCGTTTT | ACCTCCCGAAAA | 123 | 60 |
| | | GTAAGAATCGGC | AACGCTATCG | | |
| −0.9 kb | U | TATTTTGTTGG | AACTCCAAATCAA | 96 | 58 |
| | | GTTTTTATGGT | TTCACAACATCA | | |
| | M | TATTTTGTCGG | AAATCGATTC | 90 | 58 |
| | | GTTTTTACGGC | GCGACGTCG | | |
| KIAA1752, | U | TAATGGTTTTTGA | CACAAACTATTAT | 103 | 58 |
| +0.4 kb | | GGATTGAGATTG | CAACCAATCACA | | |
| | M | TAATGGTTTTTG | CACAAACTATTAT | 103 | 62 |
| | | AGGATTGAGATC | CAACCGATCACG | | |
| FLJ43855, | U | TGGTTGTTATT | CTAAACCACACT | 88 | 56 |
| −1.1 kb | | TGGGGTGGTTG | AAAAACAAACA | | |
| | M | TGGTTGTTATT | CTAAACCACACT | 88 | 58 |
| | | TGGGGCGGTC | AAAAACGAACG | | |
| RUNX2, | U | GGTTTTGGAAAT | AAACAACAAATC | 96 | 58 |
| −0.7 kb | | TGTATATGGTGT | TCAAACCTACA | | |
| | M | TTTCGGAAATT | AACAACGAATC | 93 | 58 |
| | | GTATACGGCGC | TCGAACCTACG | | |
| −3.0 kb | U | TGTTTGAGTGTA | TCTCTCAAATCC | 123 | 59 |
| | | TATGAGTGGAT | CACAAACAACCA | | |
| | M | TGTTCGAGTGTA | TCTCTCGAATCC | 123 | 59 |
| | | TATGAGTGGAC | CACAAACGACCG | | |
| −3.8 kb | U | AGGTTTAGTTA | CCACTAAATA | 113 | 59 |
| | | GTTTTAGTTG | CCCTAACAACA | | |
| | M | AGGTTTAGTTA | CCACTAAATA | 113 | 59 |
| | | GTTTTAGTCG | CCCTAACAACG | | |
| +1.6 kb | U | GTTTGAGGGTGG | ACTACCCCAAA | 127 | 59 |
| | | GTGGTAGTTGT | AAATCTAAATCA | | |
| | M | GTTTGAGGGCGG | ACTACCCCGAAA | 127 | 59 |
| | | GTGGTAGTCGC | AATCTAAATCG | | |
| MUC8, | U | GGTAGGAGTTAT | AATACAAACACT | 140 | 55 |
| 2.0 kb | | TAGGAGAGTATT | CACCACCTAACCA | | |
| | M | GGTAGGAGTTAT | AATACAAACGCT | 140 | 60 |
| | | TAGGAGAGTATC | CACCGCCTAACCG | | |
| ESR2, | U | TTTTTTTTAAG | ACTAAAAATAC | 111 | 56 |
| 0.9 kb | | GATTTTGTGTGT | ACATTCCACCA | | |
| | M | TTTTTTTAAGG | CCAACTAAAAAT | 113 | 58 |
| | | ATTTCGCGCGC | ACACGTTCCACCG | | |
| E2F4, | U | GTGGTTAGGA | AACCCAACCT | 106 | 58 |
| 0 kb | | ATGGAAGTG | CCACCATCA | | |
| | M | GGCGGTTAGG | AACCCGACCT | 106 | 58 |
| | | AACGGAAGC | CCGCCATCG | | |
| TNFRSF14, | U | GAATTTTGTGATT | CTCTAAACAAAC | 115 | 58 |
| −0.6 kb | | TATGTGATGATG | ACAAACAATACA | | |
| | M | GAATTTCGTGAT | CTCTAAACAAAC | 115 | 58 |
| | | TTACGTGACGAC | ACAAACGATACG | | |
| SERPINB5, | U | GAATATTTTATTT | AAAAAACCTC | 111 | 56 |
| −0.3 kb | | TTTGGTTTTGTG | CAACATATTCA | | |
| | M | TTATTTTTC | AAAAAACCTC | 104 | 54 |
| | | GGTTTTGCG | CAACATATTCG | | |
| ANGPTL7, | U | GTAATAGTAAGT | CCTACAAAAAT | 120 | 58 |
| +0.5 kb | | GTATGGAGTTGT | CTAAATAACCA | | |
| | M | GTAATAGTAAGC | CCTACGAAAAT | 120 | 58 |
| | | GTATGGAGTCGC | CTAAATAACCG | | |
| TFF2, | U | GGTAGTTGTGT | CACATAACCA | 130 | 56 |
| 0.2 kb | | TTTGTGTAGGT | ATTTTCCACA | | |
| | M | GGTAGTTGTGT | CACGTAACCG | 130 | 62 |
| | | TTTGTGTAGGC | ATTTTCCACG | | |
| BGLAP, | U | AGGGTAGGGT | AATACCTCAC | 86 | 58 |
| 0.5 kb | | TTGAGTTGTT | AATACCCCCA | | |
| | M | AGGGTAGGGT | AATACCTCGC | 86 | 58 |
| | | TTGAGTCGTC | AATACCCCCG | | |
| MSLN, | U | GGAGAGATTAGA | CATAAACTCTTA | 103 | 55 |
| 0.8 kb | | GATGATTGTTGT | TCCCCAATACA | | |
| | M | GGAGAGATTAGA | CGTAAACTCTTA | 103 | 60 |
| | | GATGATCGTCGC | TCCCCAATACG | | |
| DDX53, | U | TGGTTTTTGGG | CAAATCTACAA | 105 | 57 |
| 0 kb | | GTAATTTTTGT | CCTATTTCCCA | | |
| | M | TTTTATACGAT | CAAATCTACGA | 136 | 58 |
| | | TCGGAATTCGAC | CCTATTTCCCG | | |
| MAGEA2, | U | GTTAGGTTGT | CCAAAAAAAT | 92 | 59 |
| −0.1 kb | | TGTTTAGGGT | CACAAACCCA | | |
| | M | GCGTTTGTTTT | AAATCACGAACC | 108 | 61 |
| | | TTTTCGTCGAC | CGAATATAACG | | |

The present invention further provides a diagnostic kit for cancer containing the primer applicable for the detection of the methylation of the transitional zone.

The primers included in the kit can be every sequence that can be amplified by binding complementarily to the sequence of the transitional zone of RABGEF1, STAG, CHGB, TNFRSF14, SERPINB5, ANGPTL7, TFF2, BGLAP, MSLN, DDX53, MAGEA2, VDR, ST14, CDKN2A, MYBPC2, RUNX3, RUNX2, MLH1 or PTEN gene. And, a set of a forward and a reverse primer selected from a group consisting of those sequences listed in Table 1 are more preferred. The construction of such primers can be accomplished by the conventional method well known to those in the art and a PCR product amplified by using the primer using the diagnostic kit of the invention can be confirmed by RCR machine generally used by those in the art.

The present invention also provides a simple repeated marker group used for measuring the loss of heterozygosity (LOH) and chromosome instability.

The present invention provides 40 simple repeated sequence markers which are useful for selecting cancer related chromosomes with frequent deletions and have confirmed that these markers are very useful for classifying those chromosomal variations according to the level of the loss of heterozygosity (LOH) into 4 groups: primary, LOH-L (low), LOH-H (high) chromosomal loss and microsatellite instability (MSI). The genetic variations can be divided into high-risk genotypes (LOH-H and LOH-B) and low risk genotypes (LOH-L and MIS), which are crucial factors for the prediction of survival rate of phase 2 and phase 3 stomach cancer patients (FIG. 4, FIG. 5 and FIG. 6).

By detecting the repeated sequence instability and chromosomal loss, which are two most peculiar pathological characteristics of stomach cancer, genotypes are classified to predict metastasis and recurrence.

The above marker group is a pair of a forward primer and a reverse primer selected from a group consisting of those sequences listed in Table 2.

TABLE 2

| | | | | | | |
|---|---|---|---|---|---|---|
| | | | | | Amplicon | Hetero |
| chr | Marker | Forward | Reverse | Tm | Size | (%) |

| | | | | | | |
|---|---|---|---|---|---|---|
| 3 | D3S1597 | AATACACACAA | CCTTTTTTTCA | 56 | 80-100 | 80 |
| | | ATGTCTCTCCC | GTGGTATGC | | | |
| 3 | D3S1552 | ATTCCATACT | GCAAATGCC | 56 | 140-160 | 62 |
| | | GTAGGGAGTGT | ATGCTGTA | | | |
| 3 | D3S1312 | CTTCTCACTG | GGCTCCCC | 56 | 148~158 | 60 |
| | | CATATGACTC | AGGGTAAG | | | |
| 3 | D3S1478 | GATGAAACTG | CTGCCAGTAAT | 62 | 109-140 | 79 |
| | | TGATAGCACC | GTAAATCTCC | | | |
| 3 | D3S1619 | GTCCTGCAA | TTGCTAGGAT | 59 | 161-171 | 60 |
| | | GACTCATTG | GGTTGTTTTC | | | |
| 4 | D4S1609 | TCTGAAAAT | CATCATTACT | 59 | 163-177 | 67 |
| | | GCCCTTGACC | GCTGGGATGC | | | |
| 4 | D4S2946 | GTCAAGAGG | ACCTGTCTGA | 59 | 104-126 | 72 |
| | | GCTGATTCTG | ACTTGCGTG | | | |
| 4 | D4S174 | GCAGTTCAAA | CATTCCTAGA | 54 | 114~134 | 77 |
| | | GATGAAAGTG | TGGGTAAAGC | | | |
| 4 | D4S391 | CACATAACTT | ACTGTTGTCA | 59 | 164-185 | 86 |
| | | CCCTTGCTGG | AATCAGGCTC | | | |
| 4 | D4S230 | GGTAAATAGG | TTAGGATGCT | 56 | 170-196 | 0 |
| | | GAAAATGACA | GACTTCACCA | | | |
| 5 | D5S519 | CTACTACCAG | ATCTGCAGTG | 59 | 114-128 | 83 |
| | | CAGCATTCTC | TGAGGCAATG | | | |
| 5 | D5S346 | ACTCACTCTAGT | AGCAGATAAGAC | 56 | 96-135 | 83 |
| | | GATAAATCGGG | AGTATTACTAGTT | | | |
| 5 | D5S409 | GGGATGAAGT | TAGGATGGCA | 59 | 138-154 | 69 |
| | | GTGGATAAAC | GTGCTCTTAG | | | |
| 5 | D5S349 | ATTTGGTTTCCA | TTACACCCACC | 62 | 140-158 | 72 |
| | | TAGAATCTGAGA | AGATTAAGCG | | | |
| 5 | D5S422 | TTAATTGATCTG | CAGAGCAAGGTC | 59 | 113-134 | 85 |
| | | GGCTGGAGAACC | CTGTCTGAAAAT | | | |
| 8 | D8S261 | TGCCACTGTC | TATGGCCCAG | 59 | 128-144 | 41 |
| | | TTGAAAATCC | CAATGTGTAT | | | |
| 8 | D8S262 | AGCTCAAAAG | GGCAACAAAG | 59 | 114-128 | 0 |
| | | CGAAGGTGAT | TGAGATCCTG | | | |
| 8 | D8S503 | CGTTTGGAAAT | TCGCTCAGAA | 56 | 107-120 | 45 |
| | | TGTCATTACC | ACAAACCAA | | | |
| 8 | D8S552 | AGGATTGTAA | GGGACTTTTT | 56 | 168-182 | 79 |
| | | TTTCCTTGC | GAAGGTTTG | | | |
| 8 | D8S277 | GATTTGTCCT | ACATGTTATGTT | 56 | 120-140 | 74 |
| | | CATGCAGTGT | TGAGAGGTCTG | | | |
| 9 | D9S157 | AGCAAGGCAA | TGGGGATGCCCA | 59 | 113-149 | 76 |
| | | GCCACATTTC | GATAACTATATC | | | |
| 9 | D9S200 | GCATTTCACAGG | CCTCTCTGC | 59 | 107-127 | 68 |
| | | AAATAATCTAAGG | ATGCCCCAG | | | |
| 9 | D9S270 | AGGTGTAGTCC | GATGTGACTGCTG | 59 | 87-101 | 71 |
| | | TTCTGGAATTT | TTAAAACTAGAG | | | |
| 9 | D9S199 | ACACATTCATA | GGGGAAAGCA | 56 | 144-164 | 77 |
| | | CCATAGCAGAGG | TTCAGACTTT | | | |
| 9 | D9S288 | AGCAACCT | AATCATCCA | 62 | 124-140 | 72 |
| | | CAACAGGG | GAAAGGCCA | | | |
| 13 | D13S267 | GGCCTGAAA | TCCCACCAT | 56 | 148-162 | 69 |
| | | GGTATCCTC | AAGCACAAG | | | |
| 13 | D13S263 | CCTGGCCTGTTA | CCCAGTCTTGG | 56 | 145-165 | 0 |
| | | GTTTTTATTGTTA | GTATGTTTTTA | | | |
| 13 | D13S135 | CCCTGTCTTCTA | CGGTTCTCAA | 59 | 168-174 | 75 |
| | | CTTCCTGTATGC | CCAGGAGAAA | | | |
| 13 | D13S286 | TGATTGTATG | TAGAGTGCAG | 56 | 80-90 | 0 |
| | | CATGCCTGTG | TGTCCAAACG | | | |
| 13 | D13S118 | TATAACTTGT | CCACAGACAT | 56 | 160-174 | 0 |
| | | GTGAGCACAG | CAGAGTCCTT | | | |
| 17 | TPS3 | AGGGATACTATT | ACTGCCACTCC | 62 | 103-135 | 90 |
| | | CAGCCCGAGGTG | TTGCCCCATTC | | | |
| 17 | D17S122 | CAGAACCACAAA | GGCCAGACAGA | 62 | 153-165 | 0 |
| | | ATGTCTTGCATTC | CCAGGCTCTGC | | | |
| 17 | D17S796 | CAATGGAACC | AGTCCGATAA | 62 | 144-174 | 82 |
| | | AAATGTGGTC | TGCCAGGATG | | | |
| 17 | D17S1358 | CCTAATTACACA | TAATATAAGACT | 52 | 148-160 | 0 |
| | | ATACTTTTGGGG | AACAAACAAATG | | | |
| 17 | D17S1566 | TTACTGAGCTG | CTCTTACCTTGC | 56 | 170-200 | 77 |
| | | TAATTCCATGAT | TGGTGAGATTG | | | |
| 18 | D18S67 | AAGGAGTAACT | CCTGCACTTGA | 56 | 113-129 | 82 |
| | | TGGGTTCCATC | TGAGATAGGC | | | |
| 18 | D18S57 | TTCAGGGTCT | AGAAGGCATT | 56 | 88-110 | 87 |
| | | TTTGAAGAGG | AAATTTTGCA | | | |
| 18 | D18S474 | TGGGGTGTT | TGGCTTTCAA | 59 | 119-139 | 82 |
| | | TACCAGCATC | TGTCAGAAGG | | | |
| 18 | D18S70 | AAGGCTGA | GGAATGTCAAGA | 0 | 111-126 | 0 |
| | | NCTCTACCG | AGTACCTACCATA | | | |
| 18 | D18S58 | GCTCCCGG | GCAGGAAATC | 59 | 144-160 | 0 |
| | | CTGGTTTT | GCAGGAACTT | | | |

The present invention also provides a diagnostic kit for cancer containing the simple repeated sequence marker group.

The primers included in the kit are a pair of a forward primer and a reverse primer selected from a group consisting of those sequences listed in Table 2. The construction of such primers can be accomplished by the conventional method well known to those in the art and a PCR product amplified by using the primer in the diagnostic kit of the invention can be confirmed by RCR machine generally used by those in the art.

The present inventors confirmed that methylation is increased with the increase of chromosomal loss and decreased with the reduction of chromosomal loss. Thus, investigation on methylation and measuring the level of LOH for the same lesion lead to the reduction of chances of inaccuracy resulted from the discontinuity of chromosomal loss. The result from the genetic method provided by the present invention, which was the prediction of lymph node metastasis, was more accurate than that from CT. The genetic diagnosis before surgical operation can provide information on the course of stomach cancer. Therefore, the method for genetic diagnosis of the present invention is very useful for planning operations and treatments.

### DESCRIPTION OF DRAWINGS

The application of the preferred embodiments of the present invention is best understood with reference to the accompanying drawings, wherein:

FIG. 1 is a schematic diagram illustrating that methylation is differently induced in the transitional zone located in between CpG island and a retroelement neighboring transcription starting point according to the transcription density of repeated sequences in cells or tissues, indicating the tissue specific methylation with variations. Cancer progress would be predicted by detecting the epigenetic marker in this variable region.

FIG. 2 is a block diagram illustrating that chromosome with LOH is quantified and the size of lesion is measured. Based on that, chromosomal loss is classified into LOH-H (high, 4˜8 loss) and LOH-L (low, 0˜3 loss) in the intestinal type and LOH-H (high, 4˜8 loss), LOH-L (low 2˜3 loss) and LOH-B (basal, 0˜1 loss) in the diffuse type. This classification is helpful for the comparison of genotypes between genes obtained from the endoscopy tissue and genes obtained from the operation tissue. Based on the result of the genotype comparison and measurement of the size of the cancer lesion, cancer is diagnosed before surgical operation by using the LOH.

FIG. 3 is a photograph showing the results of PCR and electrophoresis examining normal tissues and tumor tissues of each low risk group (case 10) and high risk group (case 25) exhibiting chromosome instability by using variable region epigenetic markers.

Case 10: low risk group (with chromosome instability) Case 25: high risk group (without chromosome instability but with LOH-H)

U: result of PCR using demethylation markers

M: result of PCR using methylation markers

%: degree of methylation

FIG. 4 is a photograph showing the results of PCR and electrophoresis with normal and tumor tissues by using 40 simple repeated sequence markers located at 4p, 5q, 9p, 13q, 17p and 18q. * indicates the region with chromosomal loss confirmed by comparing with normal tissues and case 25 shows LOH-H.

FIG. 5 is a set of graphs showing the survival curves obtained from 130 stomach cancer patients having operation in phases 2 and 3. When the patients are classified by genotype, the low risk group (LOH-L, chromosome instability) exhibits high survival rate, while the high risk group (LOH-H, LOH-B) exhibits low survival rate.

FIG. 6 is a set of graphs showing that chromosomal loss and the methylation of the transitional zone can be indexes for diagnosing cancer, by which high risk group (LOH-H, LOH-B) and low risk group (LOH-L, MSI) are clearly divided.

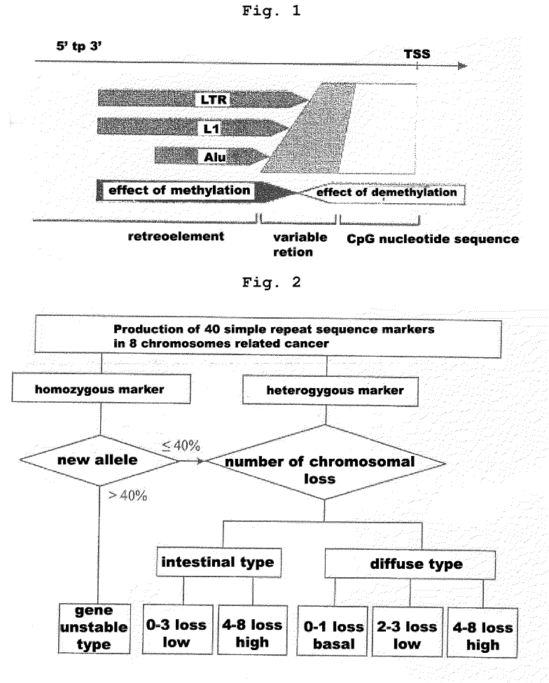

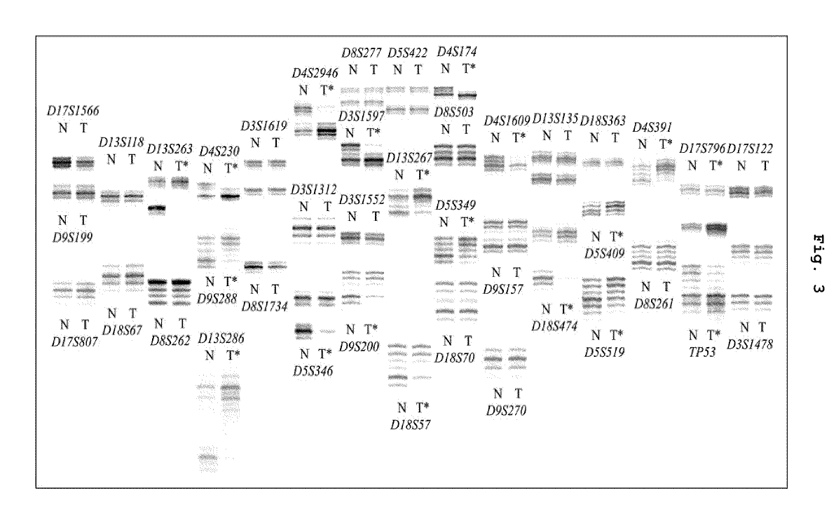

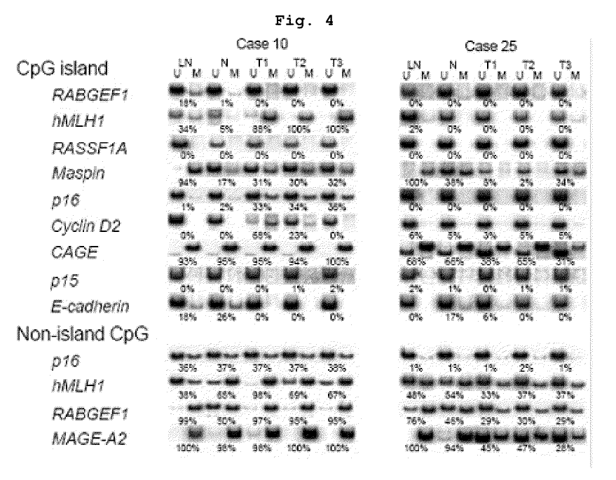

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Cancer Diagnosis Using PCR Markers Measuring the Methylation of the Transitional Zone

### <1-1> Micro-Dissection and DNA Extraction

A tumor tissue sample fixed in paraffin block was cut into 5 μm thick, and wax was eliminated by xylene, followed by hydration in ethanol. The sample was stained with hematoxylin-eosin, which was then fixed on the slide glass.

With observing under the microscope, tumor tissues and normal tissues on the slide glass were separated by using a needle. Each tissue obtained from both normal and tumor region was put in lysis buffer, which stood at 37° C. for 3 hours and then at 50° C. for 3 hours. The sample was heated before PCR to inactivate protease K.

### <1-2> Bisulfite Modification and Methylation Specific PCR for Obtaining Samples for Analysis of Methylation Pattern

NaOH was added to the DNA obtained in Example <1-1>, which stood at 37° C. for 10 minutes. Hydroquinone and sodium bisulfite (pH 5.0) were added thereto, followed by stirring. Mineral oil was loaded on the mixture, followed by reaction for 16 hours at 50° C. Purification was performed by using wizard DNA purification resin (Promega, USA), followed by elution. NaOH was added to the reaction mixture at the level of 0.3 M, followed by reaction at room temperature for 5 minutes, leading to the completion of modification. Ethanol precipitation was performed by adding ethanol in the presence of glycogen and sodium acetate, which was stirred and stood overnight at −20° C. Centrifugation was performed for 30 minutes for precipitation, followed by washing with 70% ethanol. The prepared sample was used directly or stored at −20° C. for further use.

PCR reaction mixture included 1× PCR buffer, dNTPs, P³²-dTTP, primers and bisulfite-modified DNA or unmodified DNA. Hot-start was performed at 95° C. for 5 minutes, to which Taq polymerase was added for amplification. Final extension was performed at 72° C. for 10 minutes. PCR product was loaded on polyacrylamide gel, followed by electrophoresis. Radioluminograph scanner (BAS 2500, Fuji Photo Film, Japan) was used for observation. The level of methylation (%) was calculated by using standard calibration curve and represented by percentage. In FIG. 3, U indicates the result of amplification using demethylation markers, M indicates the result of amplification using methylation markers and % indicates the degree (level) of methylation.

FIG. 3 shows the results of PCR and electrophoresis examining normal tissues and tumor tissues of each low risk group (case 10) and high risk group (case 25) exhibiting chromosome instability by using variable region epigenetic markers and markers prepared from the nucleotide sequence around CpG island. When the markers prepared from nucleotide sequence around CpG island were used, the methylation patterns of normal tissues and tumor tissues were not much different. However, when the markers prepared from the transitional zone out of CpG island sequence region were used, low risk group (case 10) showed methylation but high risk group (case 25) showed demethylation. The above results indicate that the difference of the markers of CpG island sequence region was not significant according to normal or tumor tissues, but the markers of the transitional zone is more sensitive to methylation, making the transitional zone markers for better candidates for cancer diagnosis. In high risk group (case 25), the methylation patterns of those markers of CpG island such as P15 or RASSF1A were similar between normal tissues and cancer tissues, while the methylation patterns of those markers such as MLH1 or MAGEA2 of the transitional zone between normal and tumor tissues were significantly different. In high risk group (case 25), the methylation of MLH1 (−0.6 kb) of CpG island sequence region and chromosomal loss were not significantly different between normal and tumor tissues, indicating that the MLH1 is a less preferable marker for cancer diagnosis. However the methylation of the transitional zone of the invention was significantly different between normal and tumor tissues.

### Example 2

### Measurement of LOH (Loss of Heterozygosity) by Simple Repeated Sequence Markers

PCR and electrophoresis were performed with normal and tumor tissues (stomach cancer) by using 40 simple repeated sequence markers located at 4p, 5q, 9p, 13q, 17p and 18q. As a result, at least 40% of homozygous markers (15 in total) exhibited high chromosome instability in case 10, while LOH-H chromosomal loss was detected in case 25.

### Example 3

### Comparison of Chromosomal Loss Between the Endoscopy Lesion and the Operation Lesion

As long as the endoscopy lesion can represent the total genotype, loss of heterozygosity (LOH) of the endoscopy lesion can be used for genetic diagnosis before surgery. 91 pairs of the endoscopy lesion and the operation lesion were compared and as a result 96% exhibited similar genotypes, suggesting that genetic diagnosis of the endoscopy lesion is possible in stomach cancer patients before operation.

### Example 4

### Accuracy of the Prediction of the Operation Tissue Confirmed by Combinational Assay of LOH Level and the Size of a Lesion

From the multifocal study on 130 stomach cancer patients, it was confirmed that lymph node metastasis was frequent in high risk genotype regardless of the size of a lesion. The lymph node metastasis was 83% in at least 5 cm lesion of low risk group. Intestinal invasion was frequent in larger lesion (>5 cm) than in smaller lesion (<5 cm) regardless of genotypes. Considering genotype of the endoscopy lesion together with the size of a lesion, prediction for operation tissue can be accurate and precisely receiver operating characteristics area (Roc area) for lymph node metastasis and intestinal invasion were 0.815 and 0.685, respectively.

### Example 5

### Comparison of the Accuracy Between the Prediction of the Operation Tissue Made by Combinational Assay of the Level of LOH and the Size of a Lesion and the Result of Computer Tomography

Table 3 illustrates the examples in which the accuracy of combinational assay of LOH and lesion size was compared with the result of computer tomography. As a result, the prediction by the combinational assay of LOH and lesion size was more accurate than the result of computer tomography (intestinal invasion; ROC area=0.691 vs 0.548) or (lymph node metastasis; ROC area=0.691 vs 0.642).

TABLE 3

| Pathology | Genotype-size | | Computed tomography | | p value² |
|---|---|---|---|---|---|
| Extraserosal | No | Yes | No | Yes | |
| invasion (%) | | | | | |
| No 38 (42) | 21 (70) | 17 (28) | 23 (46) | 15 (37) | |
| Yes 53 (58) | 9 (30) | 44 (72) | 27 (54) | 26 (63) | |
| ROC area¹ | 0.691 | | 0.548 | | 0.076 |
| Lymph node | No | Yes | No | Yes | |
| metastasis (%) | | | | | |
| No 36 (40) | 25 (83) | 11 (18) | 22 (61) | 14 (39) | |
| Yes 55 (60) | 5 (17) | 50 (82) | 18 (33) | 37 (67) | |

| | | | |
|---|---|---|---|
| ROC area¹ | 0.802 | 0.642 | 0.021 |

¹Area under the ROC curve
²Comparison of ROC areas between microsatellite genotype and computed tomography.

### Example 6

### Relation of Survival Rate with a Genotype Classified According to LOH and Chromosome Instability

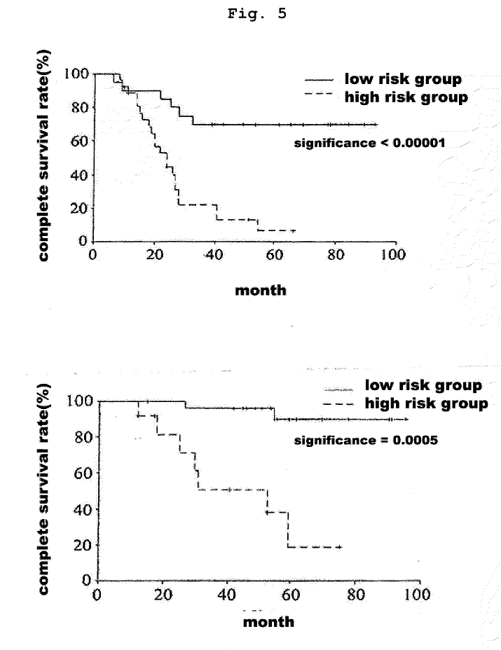

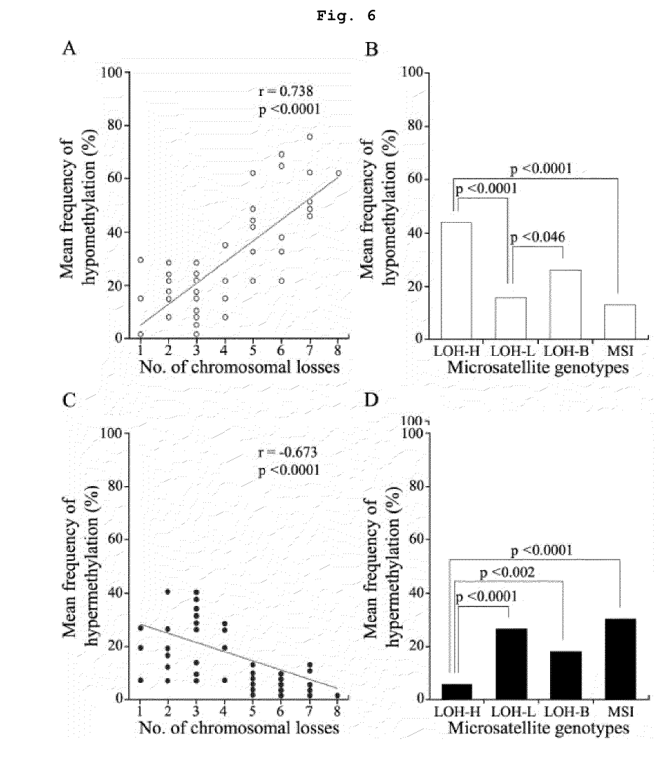

FIG. 5 is a set of graphs showing the survival curves obtained from 130 stomach cancer patients having operation in phases 2 and 3. When the patients were classified by the genotype, low risk groups (LOH-L, chromosome instability) showed high survival rate, while high risk groups (LOH-H, LOH-B) showed low survival rate.

### Example 7

### Increasing the Accuracy of the Genetic Diagnosis by Measuring Both the Level of LOH and the Level of the Methylation of the Variable Region

FIG. 6 is a set of graphs illustrating that the division of LOH into high, low, basal and chromosome instability was more clearly confirmed by measuring methylation. FIG. 6A and FIG. 6B are graphs illustrating the decrease of methylation with the decrease of chromosomal loss and FIG. 6C and FIG. 6D are graphs illustrating the decrease of methylation with the increase of chromosomal loss. In high and basal levels of chromosomal loss, methylation was reduced, while in low level of chromosomal loss and chromosome instability, methylation was increased. Using this difference of methylation according to the level of chromosomal loss enhances the liability of the prediction of cancer diagnosis. That is, methylation is closely related to the level of LOH, so that measuring methylation can reduce the inaccuracy caused by LOH discontinuity, suggesting that co-measuring the both LOH level and methylation increases accuracy and liability to the prediction of genetic diagnosis.

### INDUSTRIAL APPLICABILITY

The present invention provides a method for increasing the accuracy of cancer diagnosis and prediction by 1) providing a marker group available for cancer diagnosis by measuring the methylation of transitional zones, 2) providing a simple repeated sequence marker group in relation to LOH, and 3) co-measuring the methylation of transitional zones and the level of LOH. Accordingly, the present invention provides a method for pre-surgery genetic diagnosis that is able to predict metastasis and prognosis with a small part of lesion, an endoscopy tissue, which will be effectively used for planning the surgery and treatment for cancer.

[Sequence List Text]

Sequences represented by SEQ. ID. NO: 1˜NO: 160 are forward primers and reverse primers for 40 epigenetic markers of transitional zones which are involved in cancer diagnosis. Sequences represented by SEQ. ID. NO: 1˜NO: 4 are forward and reverse primers for RABGEF1, ˜0.2 kb, sequences represented by SEQ. ID. NO: 5˜NO: 8 are forward and reverse primers for STAG1, −0.4 kb, sequences represented by SEQ. ID. NO: 9˜NO: 12 are forward and reverse primers for CHGB, −0.3 kb, sequences represented by SEQ. ID. NO: 13˜NO: 16 are forward and reverse primers for VDR, −0.7 kb, sequences represented by SEQ. ID. NO: 17˜NO: 20 are forward and reverse primers for VDR, +1.0 kb, sequences represented by SEQ. ID. NO: 21˜NO: 24 are forward and reverse primers for ST14, −0.3 kb, sequences represented by SEQ. ID. NO: 25˜NO: 28 are forward and reverse primers for ST14, −0.8 kb, sequences represented by SEQ. ID. NO: 29˜NO: 32 are forward and reverse primers for CDKN2A, −0.1 kb, sequences represented by SEQ. ID. NO: 33˜NO: 36 are forward and reverse primers for CDKN2A, −1.5 kb, sequences represented by SEQ. ID. NO: 37˜NO: 40 are forward and reverse primers for CDKN2A, +0.8 kb, sequences represented by SEQ. ID. NO: 41˜NO: 44 are forward and reverse primers for PPARG, −0.2 kb, sequences represented by SEQ. ID. NO: 45˜NO: 48 are forward and reverse primers for MYBPC2, −1.2 kb, sequences represented by SEQ. ID. NO: 49˜NO: 52 are forward and reverse primers for MYBPC2, −0.7 kb, sequences represented by SEQ. ID. NO: 53˜NO: 56 are forward and reverse primers for RB1, −0.4 kb, sequences represented by SEQ. ID. NO: 57˜NO: 60 are forward and reverse primers for RUNX3, −0.5 kb, sequences represented by SEQ. ID. NO: 61˜NO: 64 are forward and reverse primers for RUNX3, −1.7 kb, sequences represented by SEQ. ID. NO: 65˜NO: 68 are forward and reverse primers for RUNX3, +1.0 kb, sequences represented by SEQ. ID. NO: 69˜NO: 72 are forward and reverse primers for PAX5, −1.0 kb, sequences represented by SEQ. ID. NO: 73˜NO: 76 are forward and reverse primers for MLH1, −0.6 kb, sequences represented by SEQ. ID. NO: 77˜NO: 80 are forward and reverse primers for MLH1, −1.0 kb, sequences represented by SEQ. ID. NO: 81˜NO: 84 are forward and reverse primers for CDH1, 0 kb, sequences represented by SEQ. ID. NO: 85˜NO: 88 are forward and reverse primers for PTEN, −1.4 kb, sequences represented by SEQ. ID. NO: 89˜NO: 92 are forward and reverse primers for −0.9 kb, sequences represented by SEQ. ID. NO: 93˜NO: 96 are forward and reverse primers for KIAA1752, +0.4 kb, sequences represented by SEQ. ID. NO: 97˜NO: 100 are forward and reverse primers for FLJ43855, −1.1 kb, sequences represented by SEQ. ID. NO: 101˜NO: 104 are forward and reverse primers for RUNX2, −0.7 kb, sequences represented by SEQ. ID. NO: 105˜NO: 108 are forward and reverse primers for RUNX2, −3.0 kb, sequences represented by SEQ. ID. NO: 109˜NO: 112 are forward and reverse primers for RUNX2, −3.8 kb, sequences represented by SEQ. ID. NO: 113˜NO: 116 are forward and reverse primers for RUNX2, +1.6 kb, sequences represented by SEQ. ID. NO: 117˜NO: 120 are forward and reverse primers for MUC8, +2.0 kb, sequences represented by SEQ. ID. NO: 121˜NO: 124 are forward and reverse primers for ESR2, −0.9 kb, sequences represented by SEQ. ID. NO: 125˜NO: 128 are forward and reverse primers for E2F4, 0 kb, sequences represented by SEQ. ID. NO: 129˜NO: 132 are forward and reverse primers for TNFRSF14, −0.6 kb, sequences represented by SEQ. ID. NO: 133˜NO: 136 are forward and reverse primers for SERPINB5, −0.3 kb, sequences represented by SEQ. ID. NO: 137˜NO: 140 are forward and reverse primers for ANGPTL7, +0.5 kb, sequences represented by SEQ. ID. NO: 141˜NO: 144 are forward and reverse primers for TFF2, −0.2 kb, sequences represented by SEQ. ID. NO: 145˜NO: 148 are forward and reverse primers for BGLAP, −0.5 kb, sequences represented by SEQ. ID. NO: 149˜NO: 152 are forward and reverse primers for MSLN, −0.8 kb, sequences represented by SEQ. ID. NO: 153˜NO: 156 are forward and reverse primers for DDX53, 0 kb, and sequences represented by SEQ. ID. NO: 157˜NO: 160 are forward and reverse primers for MAGEA2, −0.1 kb.

Sequences represented by SEQ. ID. NO: 161˜NO: 240 are simple repeated sequence markers available for measuring LOH and chromosome instability. SEQ. ID. NO: 161 is a forward primer and SEQ. ID. NO: 162 is a reverse primer for D3S1597, SEQ. ID. NO: 163 is a forward primer and SEQ. ID. NO: 164 is a reverse primer for D3S1552, SEQ. ID. NO: 165 is a forward primer and SEQ. ID. NO: 166 is a reverse primer for D3S1312, SEQ. ID. NO: 167 is a forward primer and SEQ. ID. NO: 168 is a reverse primer for D3S1478, SEQ. ID. NO: 169 is a forward primer and SEQ. ID. NO: 170 is a reverse primer for D3S1619, SEQ. ID. NO: 171 is a forward primer and SEQ. ID. NO: 172 is a reverse primer for D4S1609, SEQ. ID. NO: 173 is a forward primer and SEQ. ID. NO: 174 is a reverse primer for D4S2946, SEQ. ID. NO: 175 is a forward primer and SEQ. ID. NO: 176 is a reverse primer for D4S174, SEQ. ID. NO: 177 is a forward primer and SEQ. ID. NO: 178 is a reverse primer for D4S391, SEQ. ID. NO: 179 is a forward primer and SEQ. ID. NO: 180 is a reverse primer for D4S230, SEQ. ID. NO: 181 is a forward primer and SEQ. ID. NO: 182 is a reverse primer for D5S519, SEQ. ID. NO: 183 is a forward primer and SEQ. ID. NO: 184 is a reverse primer for D5S346, SEQ. ID. NO: 185 is a forward primer and SEQ. ID. NO: 186 is a reverse primer for D5S409, SEQ. ID. NO: 187 is a forward primer and SEQ. ID. NO: 188 is a reverse primer for D5S349, SEQ. ID. NO: 189 is a forward primer and SEQ. ID. NO: 190 is a reverse primer for D5S422, SEQ. ID. NO: 191 is a forward primer and SEQ. ID. NO: 192 is a reverse primer for D8S261, SEQ. ID. NO: 193 is a forward primer and SEQ. ID. NO: 194 is a reverse primer for D8S262, SEQ. ID. NO: 195 is a forward primer and SEQ. ID. NO: 196 is a reverse primer for D8S503, SEQ. ID. NO: 197 is a forward primer and SEQ. ID. NO: 198 is a reverse primer for D8S552, SEQ. ID. NO: 199 is a forward primer and SEQ. ID. NO: 200 is a reverse primer for D8S277, SEQ. ID. NO: 201 is a forward primer and SEQ. ID. NO: 202 is a reverse primer for D9S157, SEQ. ID. NO: 203 is a forward primer and SEQ. ID. NO: 204 is a reverse primer for D9S200, SEQ. ID. NO: 205 is a forward primer and SEQ. ID. NO: 206 is a reverse primer for D9S270, SEQ. ID. NO: 207 is a forward primer and SEQ. ID. NO: 208 is a reverse primer for D9S199, SEQ. ID. NO: 209 is a forward primer and SEQ. ID. NO: 210 is a reverse primer for D9S288, SEQ. ID. NO: 211 is a forward primer and SEQ. ID. NO: 212 is a reverse primer for D13S267, SEQ. ID. NO: 213 is a forward primer and SEQ. ID. NO: 214 is a reverse primer for D13S263, SEQ. ID. NO: 215 is a forward primer and SEQ. ID. NO: 215 is a reverse primer for D13S135, SEQ. ID. NO: 217 is a forward primer and SEQ. ID. NO: 218 is a reverse primer for D13S286, SEQ. ID. NO: 219 is a forward primer and SEQ. ID. NO: 220 is a reverse primer for D13S118, SEQ. ID. NO: 221 is a forward primer and SEQ. ID. NO: 222 is a reverse primer for TP53, SEQ. ID. NO: 223 is a forward primer and SEQ. ID. NO: 224 is a reverse primer for D17S122, SEQ. ID. NO: 225 is a forward primer and SEQ. ID. NO: 226 is a reverse primer for D17S796, SEQ. ID. NO: 227 is a forward primer and SEQ. ID. NO: 228 is a reverse primer for D17S1358, SEQ. ID. NO: 229 is a forward primer and SEQ. ID. NO: 230 is a reverse primer for D17S1566, SEQ. ID. NO: 231 is a forward primer and SEQ. ID. NO: 232 is a reverse primer for D18S67, SEQ. ID. NO: 233 is a forward primer and SEQ. ID. NO: 234 is a reverse primer for D18S57, SEQ. ID. NO: 235 is a forward primer and SEQ. ID. NO: 236 is a reverse primer for D18S474, SEQ. ID. NO: 237 is a forward primer and SEQ. ID. NO: 238 is a reverse primer for D18S70, SEQ. ID. NO: 239 is a forward primer and SEQ. ID. NO: 240 is a reverse primer for D18S58.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A genetic marker for cancer diagnosis, the genetic marker containing one or more transitional zones selected from a group consisting of RABGEF1 (RAB guanine nucleotide exchange factor 1), STAG (stromal antigen), CHGB (chromogranin B), TNFRSF14 (tumor necrosis factor receptor superfamily 14), SERPINB5 (serine proteinase inhibitor, clade B, member 5), ANGPTL7 (angiopoietin-like 7), TFF2 (trefoil factor 2), BGLAP (bone gamma-carboxyglutamate (gla) protein), MSLN (mesothelin), DDX53 (DEAD (SEQ ID NO: 241) box polypeptide 53), MAGEA2 (melanoma antigen family A), VDR (vitamin D (1,25-dihydroxyvitamin D3) receptor), ST14 (suppression of tumorigenicity 14), CDKN2A (cyclin-dependent kinase inhibitor 2A), MYBPC2 (myosin binding protein C, fast type), RUNX3 (runt-related transcription factor 3), RUNX2 (runt-related transcription factor 2), MLH1 (MutL DNA mismatch repair protein) and PTEN (Phosphatase and Tensin homolog deleted on chromosome Ten).

2. A method for one or both of diagnosing cancer and predicting metastasis or prognosis by measuring the DNA methylation of a tissue sample, the method comprising:
preparing a tissue sample for measuring the DNA methylation of one or more transitional zones of the tissue sample; and
measuring the DNA methylation of the one or more transitional zones.

3. The method according to claim 2, wherein the transitional zone is one or more zones selected from a group consisting of RABGEF1, STAG, CHGB, TNFRSF14, SERPINB5, ANGPTL7, TFF2, BGLAP, MSLN, DDX53, MAGEA2, VDR, ST14, CDKN2A, MYBPC2, RUNX3, RUNX2, MLH1 and PTEN.

4. A primer for detecting the DNA methylation of one or more transitional zones.

5. The primer according to claim 4, wherein the primer comprises a set of forward and reverse primers selected from a group consisting of sequences represented by SEQ. ID. NO: 1˜NO: 160.

6. A diagnostic kit for diagnosing cancer, wherein the diagnostic kit contains the primer of claim 4.

7. A simple repeated sequence marker group for measuring the loss of heterozygosity (LOH) and the level of chromosome instability.

8. The marker group according to claim 7, wherein the marker group comprises a set of forward and reverse primers selected from a group consisting of sequences represented by SEQ. ID. NO: 161˜NO: 240.

9. A diagnostic kit for diagnosing cancer, wherein the diagnostic kit contains the marker group of claim 7.

10. The method according to claim 2, further comprising measuring the level of LOH.

11. The diagnostic kit for cancer according to claim 6, wherein the kit further comprises a simple repeated sequence marker group available for measuring the loss of heterozygosity (LOH) and the level of chromosome instability.

12. The genetic marker of claim 1, wherein the cancer diagnosis comprises diagnosing stomach cancer.

13. The genetic marker of claim 1, wherein the genetic marker is used in the diagnosis of cancer.

14. The method according to claim 2, wherein the transitional zone comprises a region formed in between a CpG island and a neighboring retroelement.

15. The method according to claim 2, wherein the transitional zone is characterized by one or more of transcriptional density dependent methylation, differing levels of variability, and differences in patterns between normal and tumor tissues.

16. The method according to claim 2, wherein the cancer comprises stomach cancer.

17. The method according to clam 2, wherein the diagnosis or prediction is a preoperative diagnosis or prediction.

18. The method according to claim 17, wherein the diagnosis or prediction is made using an endoscopically-obtained tissue sample.

19. The diagnostic kit of claim 6, further comprising one or more DNA methylation reagents required to affect detection of DNA methylated transitional zones.

20. The diagnostic kit of claim 9, further comprising one or more DNA methylation reagents required to affect detection of DNA methylated transitional zones.
